(19) United States
(12) Patent Application Publication
(10) Pub. No.: US 2020/0245934 A1
(43) Pub. Date: Aug. 6, 2020

(54) STRETCHABLE ELECTRODE, METHOD FOR PRODUCING STRETCHABLE ELECTRODE, BIOLOGICAL INFORMATION MEASURING GARMENT, AND METHOD FOR MEASURING BIOLOGICAL INFORMATION

(71) Applicant: TOYOBO CO., LTD., Osaka (JP)

(72) Inventors: Haruhiko NARUSAWA, Shiga (JP); Michihiko IRIE, Shiga (JP); Takashi KONDO, Shiga (JP)

(73) Assignee: TOYOBO CO., LTD., Osaka (JP)

(21) Appl. No.: 16/639,816

(22) PCT Filed: Aug. 22, 2018
(86) PCT No.: PCT/JP2018/031004

§ 371 Date: Feb. 18, 2020

(30) Foreign Application Priority Data
Aug. 24, 2017 (JP) 2017-161251

(51) Int. Cl. A61B 5/00; H01B 1/22; H01B 13/00

(52) U.S. Cl. CPC A61B 5/6804; H01B 1/22; A61B 2562/164; A61B 2562/125; H01B 13/0036

(57) ABSTRACT

A stretchable electrode, wherein the stretchable electrode: is brought into contact with skin for measuring biological signals, has an area of 1 to 500 cm², has a thickness of 10 to 800 μm, and contains an organic compound having a molecular weight of 2,000 or less, and a content of the organic compound is 1 to 5,000 ppm The stretchable electrode is safe for use on skin, does not cause a skin rash or inflammation of the skin caused by low molecular weight organic components, and is excellent in washing durability.

### TECHNICAL FIELD

The present invention relates to a stretchable electrode which is capable of measuring a faint electric signal inside a living body when contacting the skin surface of the living body and which has low skin irritation and is useful for a biological information measuring garment.

### BACKGROUND ART

For measuring a faint bioelectric signal inside a living body in electroencephalography, electrocardiography, electromyography or the like, adhesive pad electrodes composed of a soft conductive adhesive gel having conductivity (Patent Document 1) or the like have been heretofore used. Since such adhesive pad electrodes are directly attached to the skin of a subject, the electrodes may be apt to irritate the skin, leading to development of skin rash or skin inflammation. The skin rash or skin inflammation may be caused by a low-molecular-weight unreacted monomer or a residual solvent in the adhesive pad, and thus, an adhesive electrode obtained using a prepolymer which does not contain an unreacted monomer, a residual solvent, etc. has been proposed (Patent Document 2).

In addition, low molecular weight components such as unreacted monomers and residual solvents are regarded as a problem in adhesive bandages that are attached to the skin like adhesive pad electrodes, and an effort to select drying conditions under which low-molecular-weight components in a patch are as little as possible (Patent Document 3), or an effort to use oligomers which do not contain skin-irritative low-molecular-weight components having a molecular weight of 2,000 or less (Patent Document 4) has been made.

Conventional adhesive pad electrodes enable measurement in a state of resting a subject with independent electrodes attached on the subject. On the other hand, garments on which electrodes are mounted have attracted attention with the demand for measurement of bioelectric signals over a long period of time in daily life, and extensible electrodes have been proposed which can be mounted on garments and which can closely adhere and adapt to a human body (Patent Document 5). In the Patent Document 5, an electrode is formed by drying a conductive paste containing a resin, conductive metal powder and an organic solvent. However, a high-boiling-point solvent is used, and an upper limit is imposed on the heating temperature and the time in order to suppress degradation of the resin, so that skin rash or skin inflammation may be developed when a solvent being a low-molecular-weight component remains, and is in contact with the skin for a long time. In addition, since a garment on which electrodes are mounted are repeatedly used, electrode properties may be deteriorated when the garment is washed.

### RELATED ART DOCUMENTS

### Patent Documents

Patent Document 1: JP-A-09-215668
Patent Document 2: JP-A-62-270134
Patent Document 3: JP-A-06-271461
Patent Document 4: JP-A-2003-049142
Patent Document 5: WO2016/114278

### DISCLOSURE OF THE INVENTION

### Problems to be Solved by the Invention

At the time when an extensible electrode composed of a resin and conductive metal powder is formed into a sheet, a liquid material as a precursor containing an organic solvent and a monomer is applied or printed, and then dried or cured to form the electrode into a sheet. When the drying or curing is insufficient, a large amount of low-molecular-weight organic compound such as an organic solvent and an unreacted monomer, which may cause skin rash or skin inflammation, remain in the sheet. Therefore it is necessary to perform the treatment at a higher temperature for a longer time. However, when the treatment is performed at an excessively high temperature for an excessively long time, the resin is degraded or embrittled, so that stretchability is deteriorated, leading to increase in sheet resistance and load, and consequently, electrode properties are deteriorated when the garment is repeatedly washed. Thus, there is a limit on reduction of the residual amount of organic solvents or unreacted monomers while maintaining stretchability and washing durability.

However, comparison of the residual amount of organic solvents or unreacted monomers with the result of skin irritation test has revealed that skin irritation is not influenced when a slight amount of the organic solvents or unreacted monomers remain in the sheet. And further examination has revealed that organic solvents or unreacted monomers released from the sheet influence skin irritation when the residual amount exceeds a certain threshold. On the other hand, it has been revealed that the residual amount of organic solvents or unreacted monomers is large, washing durability is influenced, and washing durability is deteriorated when the residual amount is large. The present invention has been made in view of the problems of conventional techniques, and an object of the present invention is to provide a stretchable electrode which does not suffer degradation of resin, deterioration of electrode properties and increase in cost due to drying at an excessively high temperature for an excessively long time, which is safe to the skin without developing skin rash or skin inflammation caused by low-molecular-weight components and which is excellent in washing durability.

### Solutions to the Problems

The inventor of the present invention has intensively studied to achieve such an object. As a result, they have found that the above problem can be solved by the following means, and have reached the present invention. That is, the present invention includes the following configurations [1] to [8].

[1] A stretchable electrode, wherein

the stretchable electrode is brought into contact with skin for measuring biological signals,

the stretchable electrode has an area of 1 to 500 cm²

the stretchable electrode has a thickness of 10 to 800 μm, and

the stretchable electrode contains an organic compound having a molecular weight of 2,000 or less,

a content of the organic compound is 1 to 5,000 ppm, and

a concentration of the organic compound in the garment is lower than the allowable concentration at which the organic compound affects health.

[2] The stretchable electrode according to [1], wherein

a sheet resistance of the stretchable electrode is 300 Do or less when the stretchable electrode is not extended, and

a sheet resistance increase ratio of the stretchable electrode at an extension ratio of 10% is less than 10.

[3] The stretchable electrode according to [1] or [2], wherein

a tensile elastic modulus of the stretchable electrode is 500 MPa or less, and

a load at extension is 100 N or less when the extension ratio of the stretchable electrode is 10%.

[4] The stretchable electrode according to any one of [1] to [3], wherein the sheet resistance increase ratio of the stretchable electrode after washing 50 times is less than 4.0.

[5] The stretchable electrode according to any one of [1] to [4], wherein

the stretchable electrode consists of a conductive sheet including conductive fine particles and a binder resin.

[6] The stretchable electrode according to any one of [1] to [5], wherein

the binder resin has an elastic modulus of 1 GPa or less and a rupture elongation of 200% or more.

[7] A method for producing the stretchable electrode according to any one of [1] to [6] includes:

preparing the stretchable electrode by coating or printing a conductive paste.

[8] A biological information measuring garment includes:

the stretchable electrode according to any one of [1] to [6], and

the stretchable electrode is on the side that contact with the living body.

[9] A method for measuring biological information includes:

measuring the biological information using a biological information measuring garment provided with the stretchable electrode according to any one of [1] to [6], wherein

the biological information is measured in a state where a concentration of an organic compound having a molecular weight of 2,000 or less in the garment is maintained at 20 ppm or less.

[10] A method for measuring biological information includes:

measuring the biological information using a biological information measuring garment provided with the stretchable electrode according to any one of [1] to [6], wherein

the biological information is measured in a state where a concentration of an organic compound having a molecular weight of 2,000 or less in the garment is maintained at a level lower than the allowable concentration at which the organic compound affects health.

### Effects of the Invention

The stretchable electrode of the present invention is attached to part of the inside of a garment, and the stretchable electrode is brought into contact with the skin for measuring biological signals, the stretchable electrode has an area of 1 to 500 cm², the stretchable electrode has a thickness of 10 to 800 μm, so that the surface area per volume is limited. The stretchable electrode contains an organic compound having a molecular weight of 2,000 or less, and a content of the organic compound is limited to 1 to 5,000 ppm. Thus, even when low-molecular-weight organic compound remain, the concentration thereof in the garment is low. So that even when the stretchable electrode closely adheres to a human body, the electrode is safe to the skin, and there is no risk of developing skin rash or skin inflammation caused by low-molecular-weight organic compound. Specifically, it is possible to measure biological information when the concentration of the organic compound in a space inside the garment is limited to 20 ppm.

Further, when the content of the organic compound contained in the stretchable electrode and having a molecular weight of 2,000 or less is limited to 1 to 5,000 ppm, washing durability is improved to the extent that the sheet resistance increase ratio after washing 50 times is less than 4.0.

Further, the stretchable electrode has a film resistance of 300Ω_{□} or less when the electrode is not extended, and the stretchable electrode has a sheet resistance increase ratio of less than 10 at an extension ratio of 10%. Therefore, electric signals necessary for measurement are obtained, and stable electric signals are secured even when the electrode is extended along with a fabric that is deformed when the posture is changed at the time of wearing. In addition, since the tensile elastic modulus of the stretchable electrode is 1 GPa or less, and the load at extension is 100 N or less when the extension ratio of the stretchable electrode is 10%, the stretchable electrode follows a fabric which is deformed at the time of changing a posture for putting on the garment. Further, the stretchable electrode is flexible and stretchable, and therefore does not impair wear feeling even when used in a state of being attached to the garment.

The stretchable electrode includes conductive fine particles and a binder resin. By using the conductive fine particles, electrical signals are secured, and the fine particles are fixed with the binder resin to maintain a shape as a stretchable electrode. Here, by using a binder resin having an elastic modulus of 1 GPa or less and a fracture elongation of 200% or more, a stretchable electrode having excellent stretchability is obtained, the electrode follows a fabric, so that a sense of discomfort does not occur, and thus stable electric signals are secured even when the electrode is extended along with the fabric.

### MODE FOR CARRYING OUT THE INVENTION

Hereinafter, a stretchable electrode according to an embodiment of the present invention will be described. The stretchable electrode of the present invention has a sheet-like shape having an area of 1 to 500 cm² and a thickness of 10 to 800 μm, and is attached to part of the inside of a garment. If the area is less than 1 cm², a stable electric signal cannot be secured when the electrode is displaced. If the area is more than 500 cm², ventilation in the garment is hindered, and vapor associated with humidity and released low-molecular-weight organic compound may be retained on part of the skin, resulting in skin rash or skin inflammation. If the thickness is less than 10 μm, there is a risk of increasing sheet resistance, so that the electrode is ruptured when a fabric is deformed at extension. If the thickness is more than 800 μm, the content of the organic compound contained in the stretchable electrode and having a molecular weight of 2,000 or less may be more than 5,000 ppm. And in this case, if the load at extension increases, so that when the fabric is extended, the electrode does not follow the fabric, and the wear feeling is impaired.

The content of the organic compound contained in the stretchable electrode and having a molecular weight of 2,000 or less is 1 to 5,000 ppm, and the concentration of the organic compound in the garment is lower than an acceptable concentration above which the organic compound influence the health. Consequently, development of skin rash or skin inflammation caused by low-molecular-weight organic compound is reduced. The emission rate of the organic compound is measured by the method specified in JIS A 1901 (Method for Measurement of Emissions of Volatile Organic Compounds (VOC), Formaldehyde and Other Carbonyl Compounds in Building Materials-Small Chamber Method), the method specified in JIS C 9913 (Method for Measurement of Emissions of Volatile Organic Compounds (VOC) and Carbonyl Compounds from Electronic Equipment-Chamber Method), and the like. The emission rate is measured by each method, and the concentration of the organic compound in the garment under specific conditions is calculated. In addition, as the acceptable concentration above organic compound influence the health, an acceptable chemical substance concentration recommended by Japan Association of Industrial Health, an acceptable work-environmental concentration recommended by American Conference of Governmental Industrial Hygienists (ACGIH), an acceptable concentration recommended by Occupational Safety and Health Administration (OSHA), and the like may be utilized, and when any of the values of these concentrations is different from others, it is preferable to utilize the smallest value. As long as the stretchable electrode has the above-described configuration, the stretchable electrode may have a layer structure of one or more conductive layers, or have an insulating layer on one surface and/or both surfaces of a conductive layer.

The content of the organic compound contained in the stretchable electrode of the present invention and having a molecular weight of 2,000 or less is limited to 1 to 5,000 ppm, and the sheet resistance increase ratio of the stretchable electrode at an extension ratio of 10% is less than 10. Thus, the sheet resistance increase ratio after washing 50 times is less than 4.0, and the stretchable electrode has excellent washing durability. When the content of low-molecular-weight organic compound is less than 1 ppm, the binder resin is deteriorated or embrittled during drying and curing required to reduce the content, shortage of low-molecular-weight organic compound acting as a lubricating effect occurs, and stress applied to the stretchable electrode at the time of washing ruptures the current-carrying structure of conductive fine particles reversibly changed during extension and shrinkage, so that sheet resistance increases. On the other hand, when the content of the low-molecular-weight organic compound is more than 5,000 ppm, the binder resin is plasticized by a large amount of the low-molecular weight organic compound and the physical properties are lowered, and the elongation is caused by the stress applied to the stretchable electrode during washing. As a result, the binder resin that holds the current-carrying structure of the conductive fine particles that reversibly change during the contraction process is broken, and the sheet resistance increases.

The stretchable electrode of the present invention is attached to part of the inside of a garment, and brought into contact with the skin to measure biological signals. Examples of the method for attaching the stretchable to the garment include a method in which a liquid conductive material is applied to a fabric inside a garment, and then cured or dried to form the stretchable electrode; a method in which the stretchable electrode is formed, and then stitched to a garment using a yam; and a method in which the stretchable electrode is bonded using a liquid adhesive or a hot-melt adhesive. The stretchable electrode of the present invention has electric properties such that sheet resistance when the electrode is not extended is 300Ω_{□} or less, and low enough for faint biological signals to be measured, and the sheet resistance increase ratio at an extension ratio of 10% is less than 10, so that stable electric signals are acquired even when the electrode is extended along with a fabric. If sheet resistance when the electrode is not stretched is more than 300Ω_{□}, and the sheet resistance increase ratio at an extension ratio of 10% is 10 or more, stable electric signals cannot be obtained. The stretchable electrode of the present invention has extension properties such that the tensile elastic modulus is 1 GPa or less, and the load at extension at an extension ratio of 10% is 100 N or less, so that even if a fabric is extended at the time when the electrode is used in a state of being attached to a garment, the electrode is extended along with the fabric, and thus the wear feeling is not impaired. The stretchable electrode of the present invention is prepared by applying or printing a conductive paste including conductive fine particles, a binder resin and an organic solvent.

The sheet resistance is synonymous with film resistance, and where the conductive sheet is square, the sheet resistance is defined as an electric resistance value over a range from any one side to the opposite side in a surface direction.

The conductive fine particles are metal-based fine particles and/or carbon-based fine particles. Examples of the metal-based fine particles include metal particles such as particles of silver, gold, platinum, palladium, copper, nickel, aluminum, zinc, lead and tin; alloy particles such as particles of brass, bronze, white copper and solder, hybrid particles such as particles of silver-coated copper; metal-plated polymer particles, metal-plated glass particles, and metal-coated ceramic particles. Examples of the carbon-based fine particles include graphite powder, activated carbon powder, scaly graphite powder, acetylene black, ketjen black, fullerene and carbon nanotube. The conductive fine particles may be of only one type or of two or more types. The binder resin is preferably a resin having an elastic modulus of 1 GPa or less and a rupture elongation of 200% or more, and examples thereof include thermoplastic resins, thermosetting resins or photo-curable resins, and rubbers or elastomers. Examples of the thermoplastic resin include low-density polyethylene, ethylene-vinyl acetate copolymers, polyvinylidene chloride and copolymer polyester. Examples of the thermosetting resin or photocurable resin include acrylic resins, silicon resins and polyurethane resins. Examples of the rubber or elastomer include urethane rubber, acrylic rubber, silicone rubber, butadiene rubber, nitrile rubber, isoprene rubber, styrene butadiene rubber, butyl rubber, chlorosulfonated polyethylene rubber, ethylene propylene rubber and vinylidene fluoride copolymer. Only one binder resin may be used, or two or more binder resins may be used. The blending amount of the conductive fine particles is determined with consideration given to sheet resistance and stretchability. If the volume ratio (%) of the conductive fine particles to the binder resin is large, sheet resistance decreases, leading to suppression of degradation of electric signals, but stretchability is reduced, leading to deterioration of a feeling of tingling and the feeling of fitting. On the other hand, when the volume ratio (%) of the conductive fine particles is small, stretchability increases, leading to improvement of tingling and the feeling of fitting, but sheet resistance increases, leading to degradation of electric signals. For balancing both the properties of the conductive fine particles and the binder resin, the blending amount of the conductive fine particles based on the amount of the binder resin is preferably 20 to 60% by volume.

The organic solvent to be used for the conductive paste has a vapor pressure of preferably 0.1 to 10,000 Pa, more preferably 02 to 5,000 Pa at room temperature (20° C.). If the vapor pressure of the organic solvent is low, the concentration in the garment is low even when the residual concentration is high, but the volume fraction of conductive fine particles decreases, so that sheet resistance increases, and the volume fraction of the resin decreases, so that rupture easily occurs at extension. In addition, it takes a high temperature and a long time to obtain necessary properties at the time of drying after application or printing, leading to deterioration of physical properties and increase in cost. On the other hand, when the vapor pressure of the organic solvent is high, the concentration in the garment may be high even at a low residual concentration, and the solvent is volatilized during the paste production process, application or printing, resulting in deterioration of workability.

Examples of the organic solvent having such a vapor pressure include toluene (2,900 Pa), ethylbenzene (930 Pa), benzyl alcohol (13 Pa), isophorone (40 Pa) γ-butyrolactone (150 Pa), methyl isobutyl ketone (2,100 Pa), cyclohexanone (500 Pa), n-propyl acetate (3.00 Pa), n-butyl acetate (1,200 Pa), n-pentyl acetate (650 Pa), n-dodecanol (2.4 Pa), ethylene glycol (7 Pa), ethylene glycol monobutyl ether (80 Pa), ethylene glycol monoethyl ether acetate (270 Pa), diethylene glycol (2.7 Pa), diethylene glycol monoethyl ether (13 Pa), diethylene glycol monobutyl ether (3.0 Pa), diethylene glycol dimethyl ether (330 Pa), diethylene glycol monoethyl ether acetate (5.6 Pa), diethylene glycol monobutyl ether acetate (5.3 Pa), propylene glycol monomethyl ether acetate (227 Pa), Solvesso 150 (78 Pa, Exxon Mobil Corporation), and two or more thereof may be contained if necessary. Such organic solvent is appropriately used so that the conductive paste has a viscosity suitable for application or printing.

As long as necessary properties for a stretchable conductive film are not impaired, the stretchable electrode of the present invention may contain insulating fine particles for obtaining mechanical properties, heat resistance and durability. The insulating fine particles are fine particles composed of an organic or inorganic insulating substance. Examples of the organic fine particles include resin-based fine particles such as acrylic resin fine particles, styrene resin fine particles and melamine resin fine particles. Examples of the inorganic fine particles include ceramic-based fine particles such as those of silica, alumina, zirconia, talc, silicon carbide, magnesia and boron nitride; and fine particles of salts hardly soluble in water such as calcium phosphate, magnesium phosphate, barium sulfate and calcium sulfate. The insulating fine particles may be of only one type or of two or more types. In addition, as long as necessary properties for a stretchable conductive film are not impaired, a conductive paste to be used for preparing the stretchable electrode of the present invention may contain a thixotropy imparting agent, a leveling agent, a plasticizer, a defoaming agent and the like for obtaining application and printing properties. The content of the organic solvent in the conductive paste depends on a method for dispersing conductive fine particles, a viscosity of a conductive paste suitable for a method for forming a conductive film, a drying method and the like. Using the conductive paste for forming the conductive film according to the present invention enables conductive fine particles to be uniformly dispersed in a resin by using a previously known method for dispersing fine particles in a liquid. For example, after a dispersion liquid of fine particles and a resin solution can be mixed, followed by uniformly dispersing the fine particles by an ultrasonic method, a mixer method, a three-roll mill method, a ball mill method or the like. Two or more of these methods can be used in combination.

For forming the stretchable electrode of the present invention, a conductive paste is applied or printed on a base material to form a coating film, and an organic solvent contained in the coating film is volatilized to dry the coating film, whereby a conductive film or a conductive pattern can be formed. The base material to which the conductive paste is applied is not particularly limited, but a flexible or stretchable material is preferable for taking advantage of the stretchability of the stretchable electrode. Examples of flexible base material include paper, cloth, polyethylene terephthalate, polyvinyl chloride, polyethylene and polyimide. Examples of the stretchable base material include polyurethane, polydimethylsiloxane (PDMS), nitrile rubber, butadiene rubber, SBS elastomer, SEBS elastomer, spandex fabric and knit fabric. These base materials are preferably stretchable in a surface direction. In this respect, the base material composed of rubber or elastomer is preferable. The step of applying a conductive paste on a base material is not particularly limited, and can be carried out by, for example, a coating method, a printing method or the like. Examples of the printing method include a screen printing method, a lithographic offset printing method, an inkjet method, a flexographic printing method, a gravure printing method, a gravure offset printing method, a stamping method, a dispensing method and squeegee printing. The step of heating the base material to which the conductive paste is applied be carried out in the air, a vacuum atmosphere, an inert gas atmosphere, a reducing gas atmosphere or the like, low-molecular-weight components are volatilized, curing reaction proceeds in some cases, and the sheet resistance and the stretchability of the electrode after curing are improved. The drying conditions in the air vary depending on a drying apparatus. For example, in an air-blowing constant-temperature dryer of forced air convention type, which has an in-storage capacity of 151 L, the heating temperature is in the range of 80 to 200° C., the heating time is 30 to 90 minutes, and a combination of the heating temperature and the heating time is selected from combinations of a low temperature and a long time and combinations of a high temperature and a short time with consideration given to the sheet resistance and the stretchability of the electrode, the concentration of low-molecular-weight components in the garment, the heat resistance of the binder resin, the vapor pressure of the organic solvent, and the like. If the heating temperature is lower than 80° C. and the heating time is less than 30 minutes, low-molecular-weight components in the coating film remain in an amount of more than 5,000 ppm, desired sheet resistance, stretchability and washing durability cannot be obtained, and the concentration of the low-molecular-weight components in the garment increases. If the heating temperature is higher than 200° C. and the heating time is more than 90 minutes, the concentration of low-molecular-components in the coating film is less than 1 ppm, so that desired stretchability and washing durability cannot be obtained because the binder resin and the base material are degraded or crosslinked, and costs are increased.

A biological information measuring garment of the present invention has a configuration in which the stretchable electrode of the present invention is mounted on part of the inside of a garment. The base material of the biological information measuring garment of the present invention is not particularly limited as long as it is a strip-shaped material such as a belt or a brassiere, and/or a garment composed of a knitted or woven fabric or a nonwoven fabric, and a stretchable garment is preferable from the viewpoint of the fitting property to a body in wearing, followability in exercise and movement, or the like for measuring biological information. Such a biological information measuring garment serves as means for measuring the biological information of a wearer, ensures a normal wearing method and wear feeling, and enables various kinds of biological information to be conveniently measured only by wearing the garment.

### Examples

Specific examples of the present invention will now be described, which should not be construed as particularly limiting the present invention.

[Preparation of Conductive Paste]

Using the materials shown in Table 1, the resin was dissolved in each solvent at a weight ratio as shown in Table 2, silver particles were added into the resulting solution, and the mixture was stirred with a three-roll mill to obtain a conductive paste.

TABLE 1

| Materials | Abbreviation | Substances |
|---|---|---|
| resin | CSM | chlorosulfonated polyethylene rubber |
| | | CSM-TS530 manufactured by Tosoh |
| | | Corporation |
| | DN3 | nitrile rubber Nipol DN003 manufactured |
| | | by Zeon Corporation |
| | UR6 | polyester-urethane resin Vyron UR6100 |
| | | manufactured by Toyobo Co., Ltd. |
| solvent | CHX | cyclohexane (vapor pressure: 500 Pa |
| | | (20° C.), acceptable concentration: |
| | | 20 ppm (ACGIH-TWA)) |
| | IPH | isophorone (vapor pressure: 40 Pa |
| | | (20° C.), acceptable concentration: 5 ppm |
| | | (ACGIH-STEL)) |
| | SOL | solvesso#150 (vapor pressure: 78 Pa |
| | | (20° C.), acceptable concentration: 17 ppm |
| | | (ExxonMobil-STEL)) |
| conductive fine | G35 | aggregated silver powder G-35 |
| particles | | manufactured by DOWA Electronics |
| | | Materials Co., Ltd. |
| | FA3 | flake-formed silver powder FA-D-3 |
| | | manufactured by DOWA Electronics |
| | | Materials Co., Ltd. |
| hot-melt sheet | UH | polyurethane-based hot-melt Elphan UH |
| | | manufactured by Nihon Matai Co., Ltd. |
| shirt | Polyester | Cool-One polyester 100% manufactured by |
| | | Sanwa Co., Ltd |

TABLE 2

| Item | | PS01 | PS02 | PS03 |
|---|---|---|---|---|
| resin | CSM | 20 | — | — |
| [parts by mass] | DN3 | — | 20 | — |
| | UR6 | — | — | 20 |
| solvent | CHX | — | — | 25 |
| [parts bv mass] | IPH | — | 45 | — |
| | SOL | 45 | — | 20 |
| silver particles | G35 | 80 | 60 | 60 |
| [parts by mass] | FAD3 | — | 20 | 20 |

[Preparation of Stretchable Electrode]

The conductive paste was applied onto a release-treated PET film with an applicator in such a manner that the dry thickness was about 100 μm, and drying was performed in an air-blowing constant-temperature drying machine at a temperature for a time as shown in Table 3, followed by peeling off the release-treated PET film to obtain a sheet-like stretchable electrode. Using this sheet, the thickness, the sheet resistance when the electrode was not extended, the sheet resistance increase ratio at extension, and the load at extension were measured by the methods described below. The measurement results in examples are shown in Table 3.

[Preparation of Garment Provided with Electrodes]

A hot-melt sheet as shown in Table 1 and a release paper were superposed on the prepared stretchable electrode with a release-treated PET film, and bonded to the stretchable electrode using a roll laminating machine with a rubber roll temperature adjusted to 120° C. In this way, an adhesive stretchable electrode sheet was obtained. This stretchable electrode sheet was cut to a wiring width of 10 mm and a wiring length of 140 mm. The release paper was peeled from the cut electrode with wiring, disposed at a predetermined position on the back side of a shirt (100% polyester), and thermocompression-bonded to the shirt with a clothes iron, and the release-treated PET film was peeled off to prepare a shirt provided with an electrode on the back side.

TABLE 3

| | | | Example | Example | Example | Comparative | Comparative | Comparative |
|---|---|---|---|---|---|---|---|---|
| | | unit | 1 | 2 | 3 | Example 1 | Example 2 | Example 3 |
| conductive paste | | — | PS1 | PS2 | PS3 | PS1 | PS2 | PS3 |
| drying | heating temperature | ° C. | 150 | 150 | 120 | 120 | 220 | 60 |
| conditions | treatment time | min | 60 | 60 | 75 | 20 | 30 | 120 |
| sheet | thickness | μm | 93 | 96 | 102 | 92 | 94 | 110 |
| properties | sheet resistance | Ω_{□} | 109 | 62 | 43 | 121 | 73 | 410 |
| | sheet resistance increase | — | 2.1 | 1.5 | 1.6 | 1.9 | 22.0 | 12.0 |
| | ratio at an extension | | | | | | | |
| | ratio of 10% | | | | | | | |
| | unit load | N/cm | 42 | 60 | 65 | 37 | 156 | 20 |
| | at an extension ratio | | | | | | | |
| | of 10% | | | | | | | |
| washing | sheet resistance | Ω_{□} | 164 | 112 | 95 | 630 | 1,037 | 1,763 |
| durability | after washing 50 times | | | | | | | |
| | sheet resistance increase | — | 1.5 | 1.8 | 2.2 | 5.2 | 14.2 | 4.3 |
| | ratio | | | | | | | |
| residual | concentration in sheet | ppm | 1,970 | 2,870 | 110 | 12,200 | * | 21,500 |
| solvent | emission rate | μg/m²/h | 0.04 | 0.04 | * | 0.30 | * | 0.40 |
| | concentration in | ppm | 3.1 | 3.4 | * | 23.5 | * | 38.8 |
| | garment | | | | | | | |
| | allowable concentration | ppm | 17 | 5 | 17 | 17 | 5 | 17 |
| skin | skin irritation index | — | 2.2 | 4.3 | 0.0 | 13.0 | 0.0 | 17.4 |
| irritation | safety evaluation | — | safe | safe | safe | acceptable | safe | improvement- |
| | | | product | product | product | product | product | needed |
| | | | | | | | | product |

* under limit of determination

[Extension Test and Measurement of Sheet Resistance]

The prepared stretchable electrode sheet was cut to a width of 10 mm and a length of 140 mm to prepare a test piece. In an extension tester (hand-drawing stretching machine) provided with two 2.5 cm-wide chucks, the cut test piece was held between the chucks with the chuck-to-chuck distance set to 5.0 cm, and the test piece was extended to an extension ratio of 10% in a longitudinal direction (displacement amount: 0.5 cm). Using a digital multimeter (“YOKOGAWA TY530” manufactured by YOKOGAWA Meters & Instruments Corporation), resistance values (fl) before and after extension were measured outside the two opposed chucks (measurement distance: 10 cm) to obtain sheet resistance (Ω_{□}) as sheet resistance before and after the test. The resistance value was measured immediately after extension (3 seconds or less after extension).

[Measurement of Load at Extension]

In a tensile tester (“RT Square M50” manufactured by ORIENTEC CORPORATION), a load (N) applied at the time when a stretchable conductive material sheet having a width of 30 mm and a test length of 50 mm was extended to an extension ratio of 10% was measured to obtain a unit load (N/cm) per 1 cm of sheet length.

[Washing Durability]

Washing conditions were set in accordance with JIS L 0844. Specifically, using a washing machine, a laundry net and a detergent (Attack manufactured by Kao Corporation), the prepared shirt provided with an electrode was washed five times in a row, and then dried in shade once, and this operation was repeated ten times. The sheet resistance of the stretchable conductor sheet after washing was measured, and a change from the initial sheet resistance (sheet resistance after washing/initial sheet resistance) was determined

[Measurement of Residual Solvent Concentration in Stretchable Electrode Sheet]

About 1 mg of a sample was collected from the prepared stretchable electrode sheet, and accurately weighed, the sample was heated twice at 220° C. for 20 minutes by thermal desorption-GCMS, and the total of the two quantitative values was determined, and defined as a residual amount Table 3 shows the measurement results in examples and comparative examples.

[Primary Irritation Test on Skin]

On the basis of SEK 48-Hour Human Closed Patch Testing, the following primary irritation test on skin was conducted. The stretchable electrode was cut to a 0.8 cm square, and applied to the back of each of a total of 23 Japanese male and female subjects, and an adhesive bandage for patch testing was attached onto the stretchable electrode. 30 to 60 minutes after the adhesive bandage was removed 48 hours after attachment and about 24 hours after the adhesive bandage was removed 72 hours after attachment, a skin symptom was visually examined to perform evaluation. The evaluation criteria were set as follows: score 0.0 for no response; score 0.5 for faint erythema; score 1.0 for clear erythema, score 2.0 for erythema and edema or papule; score 3.0 for erythema and edema/papule and small blisters; and score 4.0 for large blisters. A score for each subject was determined, and a skin irritation index was determined from the following expression (1).

Skin irritation index=total score/number of subjects×100 (1)

Further, on the basis of the determined skin irritation index, safety was evaluated from Skin Irritation Index Classification of Cosmetic Products in FY 1995 (safe product: 5.0 or less; acceptable product: 5.0 to 15.0; improvement-needed product: 15.0 to 30.0; and dangerous product 30.0 or more). Table 3 shows the results in examples and comparative examples.

[Measurement of Emission Rate of Low-Molecular-Weight Organic Compound]

In accordance with JIS A 1901 (Method for Measurement of Emissions of Volatile Organic Compounds (VOC), Formaldehyde and Other Carbonyl Compounds in Building Materials-Small Chamber Method), an emission test of low-molecular-weight organic compound was conducted in the following manner. Two square samples of 15 cm×15 cm were cut out from the prepared stretchable electrode, placed in a small stainless steel chamber having a capacity of 20 L, held at a temperature to 28° C., a relative humidity of 50% and a ventilation frequency of 0.5 times/h, and collected in a collection tube after one day, the concentration of an organic solvent in the collection tube was measured by thermal desorption-GCMS, and the emission rate was determined from the following expression (2).

Emission rate (μg/m²/h)=analytical concentration (μg/m³)×ventilation frequency (times/h)×chamber volume (m³)/sample surface area (m²) (2)

[Calculation of Concentration in Garment in Wearing]

The concentration in the garment in wearing was determined in the following manner. The wearing time was set to 48 hours after attachment in the primary irritation test on skin, the size of a void between the skin and the electrode was set to 100 μm with consideration given to the irregularities of the skin and the electrode, the concentration (mg/m³) was determined from the obtained emission rate in accordance with the following expression (3), and the concentration (ppm) at 25° C. and 1 atm was determined from the following expression (4).

Concentration in garment (mg/m³)=electrode area (m²)/space volume between skin and electrode (m³)×emission rate (mg/m²/h)×wearing time (h) (3)

Concentration in garment (ppm)=24.46×concentration in garment (mg/m³/molecular weight of organic solvent (4)

In Examples 1, 2 and 3, drying is performed at an appropriate heating temperature for an appropriate treatment time to reduce the amount of a residual solvent in the sheet and the concentration of the residual solvent in the garment while satisfying sheet properties, so that a stretchable electrode having excellent washing durability and low skin irritation can be obtained. In Comparative Examples 1 and 3, the drying conditions are insufficient, so that a residual solvent with a high concentration remains in the sheet, and the emission rate increases, leading to increase in concentration in the garment. In Comparative Example 2, the excessive drying conditions cause the binder resin to be cured, so that satisfactory stretchability and washing durability cannot be obtained.

As described above, in the stretchable electrode of the present invention, the content of low-molecular-weight organic compound is limited, and the concentration of the organic compound in the garment is maintained at an acceptable concentration or a lower concentration. Thus, the stretchable electrode has no risk in safety of the skin even when closely adhering to a human body, and has excellent washing durability, the wear comfort and the wear feeling are not impaired because the load at extension is low, and biological electric signals with less electric noises can be obtained because increase in sheet resistance is suppressed at extension.

### INDUSTRIAL APPLICABILITY

The present invention provides a stretchable laminated electrode and a garment for measurement biological information, which suppress development of skin rash and skin inflammation caused by low-molecular-weight organic compound, are excellent in washing durability, wear comfort and feeling of wearing, and enables favorable electric signal measurement. The stretchable electrode and the garment measurement can be applied to health management in everyday life, grasping of biological information in outdoor sports such as jogging and marathon, and labor management in outdoor work at building sites.

1. A stretchable electrode, wherein
the stretchable electrode is brought into contact with skin for measuring biological signals,
the stretchable electrode has an area of 1 to 500 cm²,
the stretchable electrode has a thickness of 10 to 800 μm, and
the stretchable electrode contains an organic compound having a molecular weight of 2,000 or less, and
a content of the organic compound is 1 to 5,000 ppm.

2. The stretchable electrode according to claim 1, wherein
a sheet resistance of the stretchable electrode is 300Ω_{□} or less when the stretchable electrode is not extended, and
a sheet resistance increase ratio of the stretchable electrode at an extension ratio of 10% is less than 10.

3. The stretchable electrode according to claim 1, wherein
a tensile elastic modulus of the stretchable electrode is 500 MPa or less, and
a load at extension is 100 N or less when the extension ratio of the stretchable electrode is 10%.

4. The stretchable electrode according to claim 1, wherein
the sheet resistance increase ratio of the stretchable electrode after washing 50 times is less than 4.0.

5. The stretchable electrode according to claim 1, wherein
the stretchable electrode consists of a conductive sheet including conductive fine particles and a binder resin.

6. The stretchable electrode according to claim 1, wherein
the binder resin has an elastic modulus of 1 GPa or less and a rupture elongation of 200% or more.

7. A method for producing the stretchable electrode according to claim 1, comprising
preparing the stretchable electrode by coating or printing.

8. A biological information measuring garment, comprising
the stretchable electrode according to claim 1.

9. A method for measuring biological information, comprising
measuring the biological information using a biological information measuring garment provided with the stretchable electrode according to claim 1, wherein
the biological information is measured in a state where a concentration of an organic compound having a molecular weight of 2,000 or less in the garment is maintained at 20 ppm or less.
